(19) United States
(12) Patent Application Publication
(10) Pub. No.: US 2026/0015309 A1
(43) Pub. Date: Jan. 15, 2026

(54) PROCESS FOR THE SYNTHESIS OF VITAMIN K2

(71) Applicant: Synergia Life Sciences Pvt. Ltd., Mumbai (IN)

(72) Inventors: Mayank SHASTRI, Lubbock, TX (US); Mrunali H. BAIKAR, Mumbai (IN); Parin K. VORA, Mumbai (IN); Raju BNS, Hyderabad (IN)

(73) Assignee: Synergia Life Sciences Pvt. Ltd., Mumbai (IN)

(21) Appl. No.: 18/992,915

(22) PCT Filed: Jul. 10, 2023
(86) PCT No.: PCT/IB2023/057055

§ 371 Date: Jan. 9, 2025

(30) Foreign Application Priority Data
Jul. 11, 2022 (IN) 202221039642

(51) Int. Cl. C07C 46/02; C07F 7/18

(52) U.S. Cl. CPC C07C 46/02; C07F 7/1892

(57) ABSTRACT

The present disclosure relates to the synthesis of Vitamin K2 bearing varying prenyl side chains. The synthesis comprises the reaction of phenyl sulfone of tert-butyl dimethyl silyl (TBDMS) protected mono prenyl menadiol with prenyl halides bearing varying prenyl units in the presence of the phase transfer catalyst followed by reductive desulphonation to yield TBDMS ether of Vitamin K2. The TBDMS ether is then oxidized in the presence of chromium trioxide and periodic acid to yield the corresponding Vitamin K2.

### RELATED APPLICATION

This application is related to and claims priority from the Indian Provisional Application 202221039642 filed 11 Jul. 2022 and PCT application PCT/IB2023/057055 filed 10 Jul. 2023 both of which are incorporated herein in its entirety.

### FIELD OF THE INVENTION

The present disclosure relates to the synthesis of Vitamin K2 bearing varying prenyl side chains.

### BACKGROUND OF THE INVENTION

Vitamin K2s are 2-methyl-1, 4-naphthoquinone derivatives and represent a series of molecules containing a naphthoquinone structure and varying lengths of prenyl chains attached at the 3 position. Their occurrence, structure, physicochemical, pharmacological properties and actions are well documented in the literature. Although Vitamin K2s occur in nature, significant efforts continue to isolate the compounds from natural sources and also synthesize the compounds by the synthetic chemistry route as well as biotechnological routes. The chemical synthesis allows the preparation of the specific member of the Vitamin K2 series of interest with high selectivity rather than the production of a mixture of different Vitamin K2s produced by the biotechnological route and isolation of the specific member of interest from the mixture.

Amongst various members of the Vitamin K2 series, Vitamin K2-7 is of significant commercial interest, although other members of the series and their derivatives have also evoked significant interest, following a publication by Suhara et. al. (Biorganic Med. Chem Lett. 17 (2007) 1622-1625) indicating development of new drugs based on side-chain modification of the alkyl group. It also reported various syntheses of menaquinone analogues in which the terminal methyl group is converted to a hydroxyl, aldehyde or acid group.

There is also an interest in exploring Vitamin K2 type compounds for inhibitory effects on osteoporosis, as potential anti-cancer drugs and controlling cardio-vascular activity.

Isler et al Helv. Chim. Acta 1958, 41, 786-807 reported first chemical synthesis of Vitamin K2-7. In the case of Vitamin K2 group of compounds, a “lack of biological activity” was ascertained for the cis form, in the journal, J. Nutrition 105: 1519-1524, 1975. According to O. Isler et. al. Angew. Chem., 71. (1959) no. 1 pages 13-15, in the case of Vitamin K1 and K2, the mono cis compounds (cis double bond adjacent to the naphthoquinone ring system) showed a significant lower activity than the all trans form.

U.S. Pat. No. 4,089,873 (1978) described the preparation of Vitamin K2 using a copper-mediated coupling reaction. U.S. Pat. No. 4,199,531 (1980) described the preparation of quinones using aryl sulfonyl/halogen coupling chemistry. The synthesis of side chain of menadiol derivative having at position C-3 from 1 to n terminal activated isoprenyl units, was accomplished by its stereo- and regio-selective alkylation with activated side chain precursor consisting of isoprenyl units.

Naruta, J Org. Chem 1980, 45, 4097-4104, described the synthesis of Vitamin K2 analogues using trialkyl allyl stannane to link the preformed side-chain to the naphthoquinone group. U.S. Pat. No. 4,229,356 (1980) disclosed the reaction of 2 methyl 1, 4 hydro naphthoquinone with a compound selected from phytyl halide, isophytyl halide, geranyl halide, farnesyl halide, geranyl, geranyl halide, preferably bromide in a heterogeneous medium comprising a quaternary ammonium ion or a tetra alkyl phosphonium ion followed by the oxidation of the resulting hydro precursor to yield the corresponding Vitamin K2. According to the inventors of U.S. Pat. No. 4,603,223 (1986), the then known processes for the manufacture of Vitamin K2 and related compounds which started from hydroquinones or mono acylated hydroquinones were unsatisfactory, since a relatively large number of reaction steps were involved. Processes starting from menadione itself or a readily accessible derivative thereof were then unknown. The patent described the synthesis of substituted 2 methyl quinone/2 methyl naphthoquinone cyclopentadiene adduct which was then prenylated in 3 position and then the desired Vitamin K derivative was recovered by subjecting the prenylated derivative of the menadione cyclopentadiene adduct to a Retro Diels Alder reaction, wherein the stereo specificity of the prenyl side chain was maintained.

Min et al. (J. Org. Chem. 2003, 68, 7925-7927) described the Friedel-Crafts allylation of a prenyl group stabilized by a sulfone moiety. The reference further related to the synthesis of ubiquinones and menaquinones from the resulting protected p-hydroquinone containing the C5 trans-allylic sulfone moiety.

Preparing Vitamin K2 having different numbers of isoprenoid units maintaining the stereo specificity of the sidechain is a challenging task. Methods have been developed to maintain selectivity by extending one isoprenoid unit at a time (Coates et al Org. Synth. 2007, 84, 43-57). However, the yield and stereo specificity decreased each time a prenyl unit was added.

Kumada and Suzuki chemistry have been exploited to connect a prenyl side chain consisting of desired number of prenyl units to the naphthoquinone ring. The prenyl side chain consisting of desired number of prenyl units could be obtained using Biellmann chemistry.

Using this approach Vitamin K2-7 could be synthesized by either of the following methods. 1) attachment of heptaprenyl chain directly to menadiol molecule, i.e. “0+7” strategy; 2) attachment of shorter chain fragments to mono prenyl derivative of menadiol, “1+n+m” strategy; and 3) attachment of hexaprenyl chain to mono prenyl derivative of menadiol, i.e. “1+6” strategy. Other members of the Vitamin K2 family could be similarly synthesized.

WO 2010/035000 A1 (2010) disclosed the synthesis of Vitamin K2 based on the poly prenyl ring attachment to the protected activated menadiol derivative, under Grignard/Kumada or Suzuki conditions, according to “0+7 strategy”.

WO 2011117324A2 (2011) reported a new procedure for the synthesis of polyphenols, which when reacted with appropriate menadione derivatives through Kumada synthesis or Suzuki coupling led to Vitamin K2. Pentaprenyl alcohol was synthesized from diprenyl-alcohol bromide, having protected acetyl and phenylsulfonyl triprenyl groups. After each step of the process: alkylation, desulfonylation and removal of hydroxyl protecting groups, purification of the product by silica gel flash chromatography was necessary. Polyprenyl halides obtained according to this procedure were used in the synthesis of Vitamin K2-7, under Grignard/Kumada or Suzuki conditions, following “0+7” or “2+5” strategy.

Chinese patent CN 102351677A (2014) described the catalytic hydrogenation of 2 methyl 1,4 naphthoquinone to 2 methyl 1,4-hydro naphthoquinone followed by Friedel Craft's alkylation with geraniol and the oxidation of the alkylated product to yield Vitamin K2.

U.S. Pat. No. 9,012,693 (2015) described a strategy for the synthesis of Vitamin K2-7 and other menaquinones using Kumada or Suzuki chemistry to attach a side chain to the naphthoquinone ring which could be further manipulated using Biellmann chemistry to produce the desired Vitamin K2. In both cases an alkyl protected menadiol could be used. The reaction was illustrated using a methyl protected menadiol. This route to the synthesis of Vitamin K2 needed the polyprenyl compound containing the requisite prenyl units, which if not commercially available, needed to be synthesized.

Biellmann chemistry afforded the synthesis of phenythio or phenylsulfonyl substituted compounds and reaction with an electrophile, such as a halide, in the presence of a base. Two or more isoprenoid chains could be coupled to make a new isoprenoid chain containing the desired number of prenyl units making use of the double Biellmann chemistry. The chain extension reactions however necessitated the use of very low temperatures. The protected naphthoquinone was oxidized to naphthoquinone in the presence of ceric ammonium nitrate (CAN). Vitamin K2-7 could also be obtained by using Kumada coupling twice.

EP 2346806 B1 (2016) described the synthesis of novel intermediate compounds and of a compound that formed part of Vitamin K2. The patent described the synthesis of a precursor for -E poly prenyl side chains using Biellmann chemistry. This involved the formation of phenythio or phenylsulfonyl substituted compounds and reaction of these sulphur compounds with an electrophile, such as a halide, in the presence of a base. The poly prenyl unit was reacted with a protected menaquinone derivative prepared using either Kumada or Suzuki coupling reaction.

The phenythio or phenylsulfonyl derivatives were reduced using lithium metal or a metal hydride. The product was then deprotected and oxidized using ceric ammonium nitrate to obtain the desired Vitamin K2. The strategy used herein for the synthesis of Vitamin K2-7, was 2+5 strategy, which the patentees claimed yielded better stereochemistry and resulted in solid, crystalline Vitamin K2-7. Kumada or Suzuki coupling reaction yielded a Vitamin K2 containing only a short prenyl chain. By using a double Bielmann coupling or triple Bielmann coupling, Vitamin K2 s of increasing chain length could be prepared. Another benefit according to the patentees was that the selenium dioxide reduction step used to form the naphthoquinone reactant took place more readily on a naphthoquinone carrying on 2 isoprenoid units than on a longer chain molecule. The synthesis of these compounds involved multiple synthesis steps and use of very low temperatures.

U.S. Pat. No. 9,464,021 (2016) described methods for the regio and stereospecific synthesis of polyprenylated quinone derivatives, e.g., Vitamin K1, K2 and Ubiquinone using dithioacetals, especially 1, 3-dithiane,

U.S. Pat. No. 9,828,323 (2017) described a process for preparation of Vitamin K2-7 which involved a) reacting an a-sulfonyl carbanion generated in situ from the phenyl sulfone of mono prenyl derivative of alkyl protected menadiol in the presence of a strong organometallic base, with a hexaprenyl halide to yield the phenylsulfonyl derivative of menadiol b) removing the phenylsulfonyl groups from the menadiol derivative by the reductive elimination to yield the alkyl protected menadiol containing the heptaprenyl unit and c) subjecting the alkyl menadiol containing the heptaprenyl unit to oxidative deetherification, to yield Vitamin K 2-7.

More particularly phenyl sulfone of mono prenyl derivative of ethyl protected menadiol was obtained by reacting diethoxy menadiol with chloro prenyl phenyl sulphone at 0° C. in the presence of SnCl₄. This was reacted with 12-phenylsulfonyl hexaprenyl bromide in the presence of sodium bis(trimethylsilyl) amide (NaHMDS) at −20° C. to obtain phenylsulfonyl heptaprenyl dimethoxy menadiol and then with [1,2-bis(diphenyl phosphino)ethane]dichloro palladium (II) and lithium triethyl borohydride at 0° C. to yield heptaprenyl diethoxy menadiol. This on treatment with ceric ammonium nitrate yielded Vitamin K2-7. 12-phenylsulfonyl hexaprenyl bromide was obtained by the bromination of the corresponding alcohol using PBr₃ at 0° C. The alcohol in turn was obtained by multiple step synthesis starting from farnesyl acetate involving reactions at very low temperatures.

EP 2917171 (2018) described the synthesis of Vitamin K2-7 using 1+6 strategy. The patent described the synthesis of synthon A as the ethoxy protected mono prenyl menadiol, having the terminal phenylsulfonyl function in allyl moiety attached in position C-3. Synthon B was hexaprenyl halide containing a phenylsulfonyl group —SO2Ph. Coupling of synthons A and B in the alkylation reaction resulted in a Vitamin K2 derivative, possessing a phenylsulfonyl group in heptaprenyl chain and hydroxyl groups protected in the ether form. Vitamin K2-7 was obtained on the removal of phenylsulfonyl groups, deetherification and oxidation.

U.S. Pat. No. 10,472,314 (2019) claimed a process for the preparation of Vitamin K2-7 comprising converting a compound

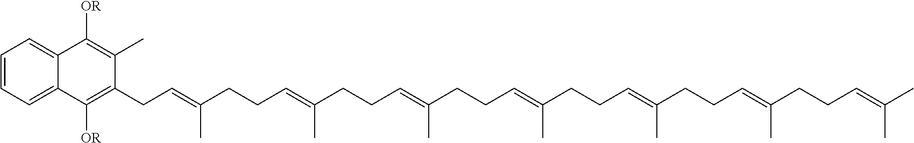

wherein R was an alkyl group; into Vitamin K2-7: wherein conversion was achieved using ceric ammonium nitrate.

EP 3 018 116 (2020) claimed the compound

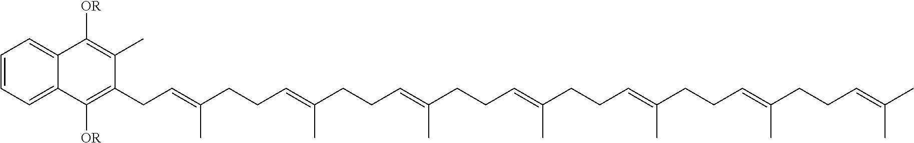

wherein R was an ethyl group i.e., the menadiol was protected by the ethyl group. The compound was then oxidized using ceric ammonium nitrate to yield Vitamin K2-7.

In view of the stability issues associated with Vitamin K2-7, efforts have been made to synthesize prodrugs of Vitamin K2-7. U.S. Pat. No. 9,512,153 B2 (2016) and U.S. Pat. No. 10,159,687 B2 (2018) described the conversion of diketone of Vitamin K2 in to a monosubstituted or disubstituted ester which was converted to Vitamin K2-7 in the body.

### SUMMARY OF THE INVENTION

A scrutiny of the prior art reveals that more emphasis is being laid on the new chemical routes for the synthesis of Vitamin K2, especially Vitamin K2-7, which would be in all trans form, involve minimal number of steps, use readily available raw materials, avoid extreme reaction conditions, maximize yield and minimize impurities. The inventors of the present disclosure have found that Vitamin K2 can be obtained by reacting the phenyl sulfone of mono prenyl derivative of TBDMS protected menadiol with prenyl halides containing varying prenyl units in the presence of phase transfer catalysts and potassium tert-butoxide at −5 to 0° C., followed by reductive desulphonation to yield TBDMS protected Vitamin K2 and subsequent oxidation in the presence of chromium trioxide and periodic acid to yield Vitamin K2. Individual members of the family can be obtained by the choice of the corresponding prenyl halide.

According to an embodiment of the invention the phenyl sulfone of mono prenyl derivative of TBDMS protected menadiol is reacted with prenyl halides containing varying prenyl units in the presence of phase transfer catalysts and a base such as potassium tert-butoxide.

According to an embodiment of the invention the number of prenyl units in the prenyl halide varies from 1 to 8.

According to an embodiment of the invention the mole ratio of phenyl sulfone of mono prenyl derivative of TBDMS protected menadiol to prenyl halide is in the range 1:1.25 to 1:1.33.

According to an embodiment of the invention the prenyl halide is a chloride.

According to an embodiment of the invention the prenyl halide is a bromide.

According to an embodiment of the invention the reaction between the phenyl sulfone of mono prenyl derivative of TBDMS protected menadiol and prenyl halide is carried out in the temperature range −5 to 0° C.

According to an embodiment of the invention the phase transfer catalyst comprises a quaternary alkyl ammonium halide and a crown ether.

According to an embodiment of the invention the phase transfer catalyst comprises tetra butyl ammonium bromide (TBAB) and 1, 4, 7, 10, 13, 16-hexa oxacyclo octadecane (18 crown 6).

According to an embodiment of the invention the mole ratio of TBAB and 18 crown 6 is varied in the range 0.5 to 20.

According to an embodiment of the invention the reaction time is varied between 0.25 to 6 hrs. According to an embodiment of the invention the sulpho phenyl prenyl derivative of TBDMS protected menadiol is reduced to yield the TBDMS protected Vitamin K2 in the presence of (1,3-bis(diphenyl phosphino) propane) palladium II chloride and lithium triethyl borohydride. According to an embodiment of the invention the TBDMS protected Vitamin K2 is oxidized in the presence of chromium trioxide and periodic acid to yield Vitamin K2.

According to an embodiment of the invention the mole ratio of periodic acid to TBDMS Vitamin K2-SO2Ph is in the range 2:1 to 4:1.

According to an embodiment of the invention the ratio of chromium trioxide to TBDMS Vitamin K2-SO2Ph is in the range 0.5 to 2 wt. %.

According to an embodiment of the invention TBDMS ethers of Vitamin K2SO2Ph are oxidized to the corresponding prenyl sulphone of menadione.

According to an embodiment of the invention the oxidation is carried out in the temperature range −5 to 0° C.

### DETAILED DESCRIPTION OF THE INVENTION

Amongst various approaches for the synthesis of Vitamin K2, Kumada and Suzuki coupling of alkyl protected menadiol with a prenyl derivatives containing desired number of prenyl units as described in EP 2346806 B1 (2016) and EP 3018116B1 (2020) is the most recent one. The latter disclosure states “The Kumada or Suzuki coupling reaction can therefore yield only a relatively short side chain which can then be built up to form a compound containing longer side chain. To complete the synthesis of a longer chain menaquinone further Kumada, Suzuki or any other chemistry can be used” It also appears that the choice of the protecting group significantly affects the reactions.

U.S. Pat. No. 9,828,323 (2017) describes a process for preparation of Vitamin K 2-7 which involves reacting an a-sulfonyl carbanion generated in situ from the phenyl sulfone of mono prenyl derivative of alkyl protected menadiol in the presence of a strong organometallic base, with a hexaprenyl halide to yield the phenylsulfonyl derivative of menadiol. Phenyl sulfone of mono prenyl derivative of ethyl protected menadiol was obtained by reacting diethoxy menadiol with chloro prenyl phenyl sulphone at 0° C. in the presence of SnCl4. This was reacted with 12-phenylsulfonyl hexaprenyl bromide in the presence of sodium bis(trimethylsilyl) amide (NaHMDS) at −20° C. to obtain phenylsulfonyl heptaprenyl diethoxy menadiol and then with [1,2-bis (diphenyl phosphino) ethane] dichloro palladium (II) and lithium triethyl borohydride at 0° C. to yield heptaprenyl diethoxy menadiol. This on treatment with ceric ammonium nitrate yielded Vitamin K2-7.

The present inventors have surprisingly found that the reaction between phenyl sulfone of mono prenyl derivative of TBDMS protected menadiol and the hexaprenyl halide can be more efficiently carried out in the presence of two-phase transfer catalysts viz. tetra butyl ammonium bromide (TBAB) and 1, 4, 7, 10, 13, 16-hexa oxacyclo octadecane (18 crown 6) to yield the phenylsulfonyl derivative of menadiol. Removal of the phenylsulfonyl groups from the menadiol derivative by the reductive elimination yields the TBDMS protected menadiol containing the heptaprenyl unit, oxidative deetherification of which in the presence of chromium trioxide and periodic acid yields Vitamin K2-7. Other members of the Vitamin K2 family can be obtained by the appropriate choice of the prenyl halide.

The invention is illustrated with the following examples which are purely illustrative in nature and do not in any way limit the scope of the invention.

### Example 1: Synthesis of t-Butyl Dimethyl Silyl (TBDMS) Ether of Menadiol: TBDMS-Menadiol: (C₂₃H₃₈O₂Si₂) (402.72)

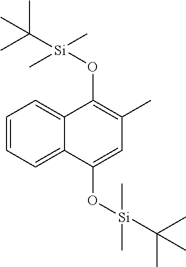 [SMILES: Cc1cc(O[Si](C)(C)C(C)(C)C)c2ccccc2c1O[Si](C)(C)C(C)(C)C]

50 g (0.29 mol) menadione was dissolved in 666 ml dichloromethane and 151.46 g (0.869 mol) sodium dithionite dissolved in 500 ml water was added. The contents were stirred for 90 minutes at room temperature. The reaction mass was filtered, the layers separated and the residue was dissolved in ethyl acetate and mixed with the methylene chloride layer. The combined organic layer was washed with water followed by brine wash and dried over sodium sulphate. The solvent was stripped off to recover menadiol (50 g).

50 g (0.287 mol) menadiol was dissolved in 500 ml tetrahydrofuran and 173.24 g (1.14 mol) tertiary butyl dimethyl silyl chloride (TBDMS-Cl), triethylamine 145.20 g (1.43 mol), 4 g TBAB catalyst and 4 g 4-dimethyl amino pyridine were added at room temperature. The reaction mass was maintained overnight at room temperature under stirring. The reaction was monitored by thin layer chromatography (TLC). After completion of the reaction, the reaction mass was quenched into 500 ml ice cold water and pH was adjusted to 7 with 1 M HCl. Tetrahydrofuran was distilled off and the residue was extracted with ethyl acetate. The layers were separated. Organic layer was washed with water followed by brine wash and dried over sodium sulphate. The solvent was distilled off and the product was isolated. TBDMS protected menadiol (123 g) was recovered.

The TBDMS-Menadiol was purified by flash chromatography. The yield of the purified product was 70%.

¹HNMR (DMSO): 8.12 (dd) 1H; 8.05 (dd) 1H; 7.45 (m) 2H; 6.71 (s) 1H; 1.15 (D) 18H; 0.93 (s) 3H, 0.31 (s) 6H, 0.21 (s) 6H. ¹³CMR (CDCl₃):145.62, 142.55, 129.06, 127.39, 125.15, 124.31, 122.74, 122.63, 122.51, 115.98, 77.39, 77.07, 76.75, 26.25, 26.03, 25.81, 25.76, 18.78, 18.50, 17.80, −2.83, −3.12, −4.15. Mass: 403.0 (m+1).

### Example 2

Synthesis of TBDMS-Vitamin-K2-3: (C₃₈H₆₂O₂Si₂)

0.5 g magnesium was activated by adding a pinch of iodine and 4 ml bromo ethane in diethyl ether (6 ml). To this activated magnesium was added at 0-5° C., 10 g (0.0207 mol) bromo derivative of TBDMS ether of menadiol dissolved in 30 ml tetrahydrofuran over 20-25 mins. Stirring was continued for 1 hr. at room temperature. The reaction mass was cooled to 0-5° C. and then 10 ml tetrahydrofuran was added followed by 3.27 g (0.0227 mol) cuprous bromide portion wise over 10-15 min. The reaction was maintained at room temperature for 1 hr. It was cooled to 0-5° C. and then 4.98 g (0.0207 mol) farnesyl chloride dissolved in 30 ml tetrahydrofuran was added. The reaction mass was maintained overnight at room temperature. The reaction was monitored by TLC. However, the reaction did not proceed.

### Example 3

Synthesis of TBDMS-Vitamin-K2-1-SO2Ph: (C₃₄H₅₀O₄Si₂) (611.01)

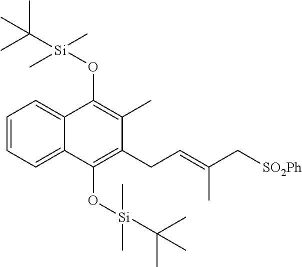

40.27 g. (0.1 mol) TBDMS-Menadiol was dissolved in 200 ml dichloromethane and 21.36 g. (0.12 mol) N-bromo succinimide (NBS) was added. The reaction mass was stirred for 1-2 hrs. After completion of the reaction, unreacted NBS was washed with sodium thiosulphate solution followed by extraction of aqueous layer with dichloromethane and subsequent drying with sodium sulphate. Stripping off the solvent yielded bromo derivative of TBDMS Menadiol in 80% yield.

2.88 g. (0.12 mol) activated magnesium turnings were taken in 20 ml tetrahydrofuran and cooled to 5-10° C., then 48.27 g. (0.1 mol) bromo derivative of TBDMS menadiol dissolved in tetrahydrofuran (124 ml) was added at the same temperature. The reaction mass was stirred for 1 hr followed by the addition of 51 ml tetrahydrofuran along with 15.8 g. (0.11 mol) cuprous bromide in portions and maintained for 1 hr. 24.4 g (0.1 mol) chloro prenyl sulphonate dissolved in 90 ml tetrahydrofuran was added dropwise over 1 hr. After completion of the reaction, the reaction mass was filtered and quenched with 25% ammonium chloride solution.

Tetrahydrofuran was recovered and the residue was extracted with methylene chloride. The organic layer was washed twice with water followed by brine and dried over anhydrous sodium sulphate. Methylene chloride was distilled under vacuum to yield 60 g crude TBDMS-Vitamin K2-1-SO2Ph. The product was purified by column chromatography in mixture of hexane and ethyl acetate to yield 42.7 g TBDMS-Vitamin-K2-1-SO₂Ph as oil.

¹HNMR (CDCl₃): 7.99 (m) 2H; 7.77 (m) 2H; 7.37 (m) 2H, 7.26 (m) 3H; 4.95 (m) 1H; 4.15 (m) 2H; 3.43 (m) 2H; 2.10 (s) 3H; 1.94 (s) 3H; 1.14 (s) 9H; 1.09 (s) 9H; 0.14 (s) 6H, 0.11 (s) 6H. ¹³CMR (CDCl₃): 171.09, 143.19, 142.57, 137.97, 134.91, 133.26, 128.76, 128.18, 127.40, 127.00, 125.57, 124.44, 124.10, 123.62, 123.10, 122.94, 122.64, 77.15, 65.98, 60.38, 31.96, 29.73, 27.36, 26.20, 26.05, 25.72, 21.06, 18.71, 18.70, 17.08, 14.49, 14.23, 14.19. Mass: 608.17 (m-3).

### Example 4

Synthesis of TBDMS-Vitamin-K2-2 (C₃₃H₅₄O₂Si₂) (538.96)

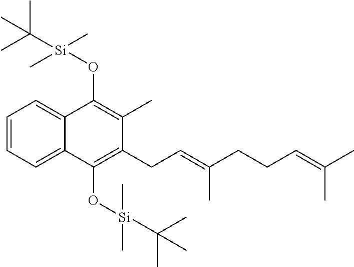 [SMILES: CC(C)=CCC/C(C)=C/Cc1c(C)c(O[Si](C)(C)C(C)(C)C)c2ccccc2c1O[Si](C)(C)C(C)(C)C]

6.11 g (˜0.01 mol) TBDMS-Vitamin-K2-1-SO₂Ph (II) was dissolved in 21 ml tetrahydrofuran and 1.85 ml dimethyl formamide under inert atmosphere. 1.3 g (˜0.0125 mol) prenyl chloride was added. The reaction mixture was cooled to 0 to −5° C. 0.266 g. (˜0.000825 mol) tetra butyl ammonium bromide (TBAB) and 0.044 g. (˜0.000165 mol) 1, 4, 7, 10, 13, 16-hexa oxacyclo octadecane (18 crown 6) was added. The reaction mass was stirred at the same temperature for 5 minutes. 1.68 g. (0.015 mol) potassium tert-butoxide in 16 ml tetrahydrofuran was added to the above solution over 30 minutes and stirred at the same temperature for 2 hrs. After completion of the reaction, the reaction was quenched by dropwise addition of 20% ammonium chloride solution and the pH was adjusted to 5-6 using 1 M HCl. Tetrahydrofuran was distilled under vacuum and the residue was extracted in ethyl acetate. Ethyl acetate layer was washed with water followed by brine and dried over anhydrous sodium sulphate. Ethyl acetate was distilled under vacuum to yield 7.3 g. crude TBDMS-Vitamin-K2-2-SO₂Ph as oil. The crude product was purified by column chromatography using the mixture of hexane and ethyl acetate to yield 4.75 g. TBDMS-Vitamin-K2-2-SO₂Ph as oil.

6.79 g. (˜0.01 mol) TBDMS-Vitamin-K2-2-SO₂Ph was dissolved in 68 ml tetrahydrofuran under inert atmosphere and 0.01 g. (˜0.000017 mol) (1, 3-bis (diphenyl phosphino) propane) palladium II chloride was added. The reaction mass was cooled to −5° C. 3.18 g. (0.03 mol) super hydride (lithium triethyl borohydride) solution was added over 30 minutes. The reaction mass was stirred at room temperature for 10-12 hrs. After completion of the reaction, it was quenched by dropwise addition of methanol followed by acetic acid and water. The reaction mass was stirred for 3 hrs and extracted twice with ethyl acetate. Ethyl acetate layer was washed with water followed by brine and dried over anhydrous sodium sulphate. Ethyl acetate was distilled under vacuum to yield 6 g. crude TBDMS-Vitamin-K2-2 as oil. Crude product was purified by column chromatography using mixture of hexane and ethyl acetate to yield 3.7 g. TBDMS-Vitamin-K2-2 as oil.

¹HNMR (CDCl₃): 8.10 (m), 2H, 7.43 (m), 2H, 5.15 (m), 2H, 3.61 (D), 2H, 2.38 (s), 3H, 2.07-2.14 (m), 4H, 1.84 (s), 3H, 1.73 (s), 3H, 1.65 (s), 3H, 1.18 (s), 18H, 0.25 (D), 12H. ¹³CMR (CDCl₃): 143.15, 142.91, 135.05, 131.31, 127.65, 127.16, 127.11, 124.39, 124.29, 123.97, 123.91, 123.12, 39.64, 27.14, 26.98, 26.69, 26.31, 26.25, 25.78, 25.62, 18.83, 18.77, 17.78, 16.45, 16.29, 15.49, 14.48, −2.81, −2.87, −3.08, −3.15, −3.36, −3.46, −4.16, Mass: 537.7 (m-1) and 556.8 (m+18).

### Example 5

Synthesis of TBDMS-Vitamin-K2-3: (C₃₈H₆₂O₂Si₂) (607.08)

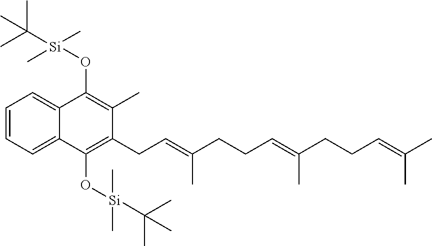 [SMILES: CC(C)=CCC/C(C)=C/CC/C(C)=C/Cc1c(C)c(O[Si](C)(C)C(C)(C)C)c2ccccc2c1O[Si](C)(C)C(C)(C)C]

6.11 g (˜0.01 mol) TBDMS-Vitamin-K2-1-SO₂Ph was dissolved in 21 ml tetrahydrofuran and 1.85 ml dimethyl formamide under inert atmosphere. 2.158 g (˜0.0125 mol) geranyl chloride was added to it. The reaction mass was cooled to 0 to −5° C. 0.266 g. (˜0.000825 mol) tetra butyl ammonium bromide and 0.044 g (˜0.000165 mol) 18 crown 6 was added. The reaction mass was stirred at the same temperature for 5 minutes. 1.68 g. (0.015 mol) potassium tert-butoxide in 16 ml tetrahydrofuran was added to the above solution over 30 minutes and the reaction mass was stirred at the same temperature for 2 hrs. After completion of the reaction, it was quenched by dropwise addition of 20% ammonium chloride solution and pH was adjusted to 5-6 using 1 M HCl. Tetrahydrofuran was distilled under vacuum and the residue was extracted in ethyl acetate. Ethyl acetate layer was washed with water followed by brine and dried over anhydrous sodium sulphate. Ethyl acetate was distilled under vacuum to yield 8 g. crude TBDMS-Vitamin-K2-3-SO₂Ph as oil. Crude product was purified by column chromatography using mixture of hexane and ethyl acetate to yield 5.2 g. TBDMS-Vitamin-K2-3-SO₂Ph as oil.

7.47 g (˜0.01 mol) TBDMS-Vitamin-K2-3-SO₂Ph was dissolved in 75 ml tetrahydrofuran under inert atmosphere and 0.01 g. (˜0.000017 mol) (1, 3-bis (diphenyl phosphino) propane) palladium II chloride was added. The reaction mass was cooled to −5° C. 3.18 g. (0.03 mol) super hydride solution was added over 30 minutes. The reaction mass was stirred at room temperature for 10-12 hrs. After completion of the reaction, it was quenched by the dropwise addition of methanol followed by acetic acid and water. The reaction mass was stirred for 3 hrs and extracted twice with ethyl acetate. Ethyl acetate layer was washed with water followed by brine and dried over anhydrous sodium sulphate. Ethyl acetate was distilled under vacuum to yield 6.9 g. crude TBDMS-Vitamin-K2-3 as oil. It was purified by column chromatography using mixture of hexane and ethyl acetate to yield 4.2 g TBDMS-Vitamin-K2-3 as oil.

¹HNMR (DMSO): 8.01 (m), 2H, 7.36 (m), 2H, 5.08 (m), 3H, 3.55 (dd), 2H, 1.94-2.29 (m), 3H, 1.76 (s), 6H, 1.68 (s), 6H, 1.57 (m), 8H, 1.13 (m), 18H, 0.16 (dd), 12H. ¹³CMR (CDCl₃): 143.10, 142.49, 135.14, 135.0, 131.22, 127.60, 127.22, 127.06, 124.43, 124.18, 124.09, 123.90, 123.52, 122.99, 122.65, 77.36, 77.04, 76.72, 39.77, 39.61, 27.09, 26.77, 26.66, 26.26, 26.19, 25.72, 18.78, 18.73, 17.70, 16.46, 15.99, 14.46, −3.13, −3.20. Mass: 607 (m-1).

### Comparative Examples

Preparation of TBDMS-K2-3-SO2Ph from TBDMS-K2-1-SO2Ph (without Phase Transfer Catalysts)

2 g (˜0.003 mol) TBDMS-Vitamin-K2-1-SO₂Ph was dissolved in 20 ml tetrahydrofuran under inert atmosphere. 0.71 g. (˜0.004 mol) geranyl chloride was added. The reaction mass was cooled to 0 to −5° C. and maintained at the same temperature for 5 minutes. 0.697 g. (0.006 mol) potassium tert-butoxide in 6 ml tetrahydrofuran was added to above solution over 10 minutes and stirred maintaining the same temperature for 24 hrs. The reaction was monitored for conversion at specific time intervals by quenching the reaction by the dropwise addition of 20% ammonium chloride solution and adjusting the pH to 5-6 using 1 M HCl. Tetrahydrofuran was distilled under vacuum and the residue was extracted in ethyl acetate. Ethyl acetate layer was washed with water followed by brine and dried over anhydrous sodium sulphate. Ethyl acetate was distilled under vacuum to yield crude TBDMS-Vitamin-K2-3-SO₂Ph as oil. The values of conversions obtained at various time intervals are summarized in the table 1 below.

Preparation of TBDMS-K2-3-SO2Ph from TBDMS-K2-1-SO2Ph (Using Only TBAB as the Phase Transfer Catalyst)

2 g. (˜0.003 mol) TBDMS-Vitamin-K2-1-SO₂Ph was dissolved in 20 ml tetrahydrofuran under inert atmosphere. 0.71 g. (˜0.004 mol) geranyl chloride was added. This was followed by adding 0.088 g. (0.27 mmol) TBAB. The reaction mass was cooled to 0 to −5° C. and stirred at the same temperature for 5 min. 0.697 g. (0.006 mol) potassium tert-butoxide in 6 ml tetrahydrofuran was added to the above solution over 10 minutes and stirred at the same temperature for 24 hrs. The reaction was monitored for conversion at specific time intervals by quenching the reaction by dropwise addition of 20% ammonium chloride solution and adjusting the pH to 5-6 using 1 M HCl. Tetrahydrofuran was distilled under vacuum and the residue was extracted in ethyl acetate. Ethyl acetate layer was washed with water followed by brine and dried over anhydrous sodium sulphate. Ethyl acetate was distilled under vacuum to yield crude TBDMS-Vitamin-K2-3-SO₂Ph as oil. The values of conversions obtained at various time intervals are summarized in the table 1 below.

Preparation of TBDMS-K2-3-SO2Ph from TBDMS-K2-1-SO2Ph (Using Only 18 Crown 6 as the Phase Transfer Catalyst)

2 g (˜0.003 mol) TBDMS-Vitamin-K2-1-SO₂Ph was dissolved in 20 ml tetrahydrofuran under inert atmosphere. 0.71 g (˜0.004 mol) geranyl chloride was added followed by the addition of 0.014 g (0.054 mmol) 18 crown 6 catalyst. The reaction mass was cooled to 0 to −5° C. and stirred at the same temperature for 5 minutes. 0.697 g (0.006 mol) potassium tert-butoxide in 6 ml tetrahydrofuran was added to the above solution over 10 minutes and stirred at the same temperature for 24 hrs. The reaction was monitored for conversion at specific time intervals by quenching the reaction by dropwise addition of 20% ammonium chloride solution and adjusting the pH to 5-6 using 1 M HCl. Tetrahydrofuran was distilled under vacuum and the residue was extracted in ethyl acetate. Ethyl acetate layer was washed with water followed by brine and dried over anhydrous sodium sulphate. Ethyl acetate was distilled under vacuum to yield crude TBDMS-Vitamin-K2-3-SO₂Ph as oil. The values of conversions obtained at various time intervals are summarized in the table 1 below.

Preparation of TBDMS-K2-3-SO2Ph from TBDMS-K2-1-SO2Ph (Using Both TBAB and 18 Crown 6 as the Phase Transfer Catalysts)

2 g. (˜0.003 mol) TBDMS-Vitamin-K2-1-SO₂Ph was dissolved in 20 ml tetrahydrofuran under inert atmosphere. 0.71 g (˜0.004 mol) geranyl chloride was added followed by 0.088 g (0.27 mmol) TBAB and 0.014 g. (0.054 mmol) 18 crown 6 catalyst. The reaction mass was cooled to 0 to −5° C. and stirred at the same temperature for 5 minutes. 0.697 g (0.006 mol) potassium tert-butoxide in 6 ml tetrahydrofuran was added to above solution over 10 minutes and stirred at the same temperature for 24 hrs. The reaction was monitored for conversion at specific time intervals by quenching the reaction by dropwise addition of 20% ammonium chloride solution and adjusting the pH to 5-6 using 1 M HCl. Tetrahydrofuran was distilled under vacuum and the residue was extracted in ethyl acetate. Ethyl acetate layer was washed with water followed by brine and dried over anhydrous sodium sulphate. Ethyl acetate was distilled under vacuum to yield crude TBDMS-Vitamin-K2-3-SO₂Ph as oil. The values of conversions obtained at various time intervals are summarized in the table 1 below.

Preparation of TBDMS-K2-3-SO2Ph from TBDMS-K2-1-SO2Ph (Using Both TBAB and 18 Crown 6 Catalysts, Varying Catalyst Ratio).

2 g. (˜0.003 mol) TBDMS-Vitamin-K2-1-SO₂Ph was dissolved in 20 ml tetrahydrofuran under inert atmosphere. 0.71 g. (˜0.004 mol) geranyl chloride was added followed by 0.176 g. (0.54 mmol) TBAB and 0.007 g. (0.027 mmol) 18 crown 6 catalyst. The reaction mass was cooled to 0 to −5° C. and stirred at the same temperature for 5 minutes. 0.697 g. (0.006 mol) potassium tert-butoxide in 6 ml tetrahydrofuran was added to above solution over 10 minutes and stirred at the same temperature for 24 hrs. The reaction was monitored for conversion at specific time intervals by quenching by dropwise addition of 20% ammonium chloride solution and adjusting the pH to 5-6 using 1 M HCl. Tetrahydrofuran was distilled under vacuum and the residue was extracted in ethyl acetate. Ethyl acetate layer was washed with water followed by brine and dried over anhydrous sodium sulphate. Ethyl acetate was distilled under vacuum to yield crude TBDMS-Vitamin-K2-3-SO₂Ph as oil. The values of conversions obtained at various time intervals are summarized in the table 1 below.

Preparation of TBDMS-K2-3-SO2Ph from TBDMS-K2-1-SO2Ph (Using Both TBAB and 18 Crown 6 Catalysts Varying Catalyst Ratio)

2 g. (˜0.003 mol) TBDMS-Vitamin-K2-1-SO₂Ph was dissolved in 20 ml tetrahydrofuran under inert atmosphere. 0.71 g. (˜0.004 mol) geranyl chloride was added followed by 0.044 g. (0.136 mmol) TBAB and 0.0285 g. (0.108 mmol) 18 crown 6 catalysts. The reaction mass was cooled to 0 to −5° C. and stirred at the same temperature for 5 minutes. 0.697 g. (0.006 mol) potassium tert-butoxide in 6 ml tetrahydrofuran was added to above solution over 10 minutes and stirred at the same temperature for 24 hrs. The reaction was monitored for conversion at specific time intervals and quenched by dropwise addition of 20% ammonium chloride solution and adjusting the pH to 5-6 using 1 M HCl. Tetrahydrofuran was distilled under vacuum and the residue was extracted in ethyl acetate. Ethyl acetate layer was washed with water followed by brine and dried over anhydrous sodium sulphate. Ethyl acetate was distilled under vacuum to yield crude TBDMS-Vitamin-K2-3-SO₂Ph as oil. The values of conversions obtained at various time intervals are summarized in the Table 1 below.

TABLE 1

| TBAB g | 18-Crown-6 g | Time hrs | % Conversion |
|---|---|---|---|
| — | — | 2 | 19..80 |
| | | 4 | 14.94 |
| | | 6 | 10.96 |
| | | 24 | o4.11 |
| 0.088 | — | 2 | 07.30 |
| | | 4 | 05.24 |
| | | 6 | 03.83 |
| | | 24 | 03.32 |
| — | 0.0142 | 2 | 17.43 |
| | | 4 | 15.89 |
| | | 6 | 14.06 |
| | | 24 | 12.49 |
| 0.088 | 0.0142 | 0.25 | 51 |
| | | 0.5 | 51 |
| | | 1 | 50 |
| | | 2 | 43.90 |
| | | 4 | 33.38 |
| | | 6 | 37.85 |
| | | 24 | 23.28 |
| 0.176 | 0.0070 | 2 | 34.82 |
| | | 4 | 24.74 |
| | | 6 | 22.17 |
| | | 24 | 08.95 |
| 0.044 | 0.0285 | 2 | 22.73 |
| | | 4 | 20.65 |
| | | 6 | 20.63 |
| | | 24 | 17.78 |

### Example 6

Synthesis of TBDMS-Vitamin-K2-4 (C₄₃H₇₀O₂Si₂) (675.20)

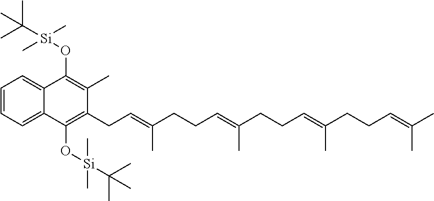 [SMILES: CC(C)=CCC/C(C)=C/CC/C(C)=C/CC/C(C)=C/Cc1c(C)c(O[Si](C)(C)C(C)(C)C)c2ccccc2c1O[Si](C)(C)C(C)(C)C]

6.11 g (˜0.01 mol) TBDMS-Vitamin-K2-1-SO₂Ph was dissolved in 21 ml tetrahydrofuran and 1.85 ml dimethyl formamide under inert atmosphere. 3 g. (˜0.0125 mol) farnesyl chloride was added to it. The reaction mass was cooled to 0 to −5° C. 0.266 g. (˜0.000825 mol) tetra butyl ammonium bromide and 0.044 g. (˜0.000165 mol) 18 crown 6 were added. The reaction mass was stirred at the same temperature for 5 minutes. 1.68 g. (0.015 mol) potassium tert-butoxide in 16 ml tetrahydrofuran was added to above solution over 30 minutes and stirred at the same temperature for 2 hrs. After completion of the reaction, it was quenched by dropwise addition of 20% ammonium chloride solution and pH was adjusted to 5-6 using 1 M HCl. Tetrahydrofuran was distilled under vacuum and the residue was extracted in ethyl acetate. Ethyl acetate layer was washed with water followed by brine and dried over anhydrous sodium sulphate. Ethyl acetate was distilled under vacuum to yield 8.5 g. crude TBDMS-Vitamin-K2-4-SO₂Ph as oil.

Crude product was purified by column chromatography using mixture of hexane and ethyl acetate to yield 5.7 g. TBDMS-Vitamin-K2-4-SO₂Ph as oil. 8.15 g (˜0.01 mole) TBDMS-Vitamin-K2-4-SO₂Ph was dissolved in 81 ml tetrahydrofuran under inert atmosphere and 0.01 g. (˜0.000017 mole) (1, 3-bis (diphenyl phosphino) propane) palladium II chloride was added. The reaction mass was cooled to −5° C. 3.18 g. (0.03 mole) super hydride solution was added over 30 minutes. The reaction mass was stirred at room temperature for 10-12 hrs. After completion of the reaction, it was quenched by the dropwise addition of methanol followed by acetic acid and water. The reaction mass was stirred for 3 hrs and extracted twice with ethyl acetate. Ethyl acetate layer was washed with water followed by brine and dried over anhydrous sodium sulphate. Ethyl acetate was distilled off under vacuum to yield 7.5 g. crude TBDMS-Vitamin-K2-4 as oil. It was purified by column chromatography using mixture of hexane and ethyl acetate to give 4.7 g. TBDMS-Vitamin-K2-4 as oil.

¹HNMR (CDCl₃): 8.04 (m), 2H, 7.39 (m), 2H, 5.11 (m), 4H, 3.55 (dd), 2H, 2.33 (s), 3H, 1.93-2.11 (m), 12H, 1.77 (s), 3H, 1.66 (s), 6H, 1.11 (m), 18H, 0.95 (m), 9H, 0.21 (dd), 12H. ¹³CMR (CDCl₃): 143.11, 142.50, 137.18, 136.89, 136.02, 135.78, 135.21, 135.17, 127.60, 127.23, 127.07, 126.87, 124.10, 124.01, 123.95, 123.91, 123.63, 123.57, 123.52, 123.47, 123.00, 122.66, 119.82, 48.97, 40.64, 40.38, 39.90, 39.69, 39.63, 35.00, 34.71, 33.19, 32.58, 31.68, 31.65, 29.87, 29.77, 29.11, 28.67, 28.47, 27.95, 27.58, 27.48, 27.10, 26.73, 26.71, 26.27, 26.21, 25.75, 25.33, 23.51, 23.09, 22.71, 19.86, 19.61, 19.25, 18.79, 18.74, 16.53, 16.29, 16.07, 16.04, 16.02, 15.90, 14.48, 14.18, 11.49, −2.89, −2.97, −3.00, −3.12, −3.19. Mass: 693 (m+18).

### Example 7

Synthesis of TBDMS-Vitamin-K2-5 (C₄₈H₇₈O₂Si₂) (743.3)

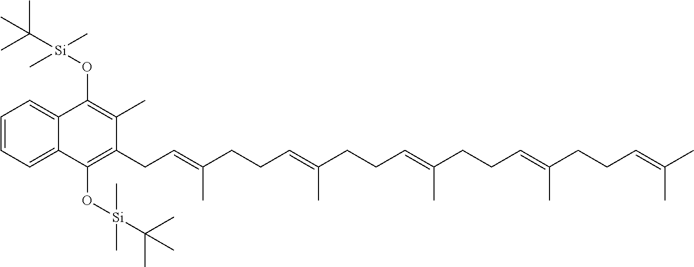 [SMILES: CC(C)=CCC/C(C)=C/CC/C(C)=C/CC/C(C)=C/CC/C(C)=C/Cc1c(C)c(O[Si](C)(C)C(C)(C)C)c2ccccc2c1O[Si](C)(C)C(C)(C)C]

6.11 g (˜0.01 mol) TBDMS-Vitamin-K2-1-SO₂Ph was dissolved in 21 ml tetrahydrofuran and 1.85 ml dimethyl formamide under inert atmosphere. 3.86 g. (˜0.0125 mol) geranyl geranyl chloride was added. The reaction mass was cooled to 0 to −5° C. 0.266 g (˜0.000825 mol) tetra butyl ammonium bromide and 0.044 g (˜0.000165 mol) 18 crown 6 were added. The reaction mass was stirred at the same temperature for 5 minutes. 1.68 g (0.015 mol) potassium tert-butoxide in 16 ml tetrahydrofuran was added to above solution over 30 minutes and stirred at the same temperature for 2 hrs. After completion of the reaction, it was quenched by dropwise addition of 20% ammonium chloride solution and the pH was adjusted to 5-6 using 1 M HCl. Tetrahydrofuran was distilled under vacuum and the residue was extracted in ethyl acetate. Ethyl acetate layer was washed with water followed by brine and dried over anhydrous sodium sulphate. Ethyl acetate was distilled under vacuum to yield 9 g. crude TBDMS-Vitamin-K2-5-SO₂Ph as oil. The crude product was purified by column chromatography using mixture of hexane and ethyl acetate to yield 6.1 g. TBDMS-Vitamin-K2-5-SO₂Ph as oil. 8.83 g (˜0.01 mol) TBDMS-Vitamin-K2-5-SO₂Ph was dissolved in 88 ml tetrahydrofuran under inert atmosphere and 0.01 g. (˜0.000017 mol) (1, 3-bis (diphenyl phosphino) propane) palladium II chloride was added. The reaction mass was cooled to −5° C. 3.18 g. (0.03 mol) super hydride solution was added over 30 minutes. The reaction mass was stirred at room temperature for 10-12 hrs. After completion of the reaction, it was quenched by the dropwise addition of methanol followed by acetic acid and water. The reaction mass was stirred for 3 hrs and extracted twice with ethyl acetate. Ethyl acetate layer was washed with water followed by brine and dried over anhydrous sodium sulphate. Ethyl acetate was distilled under vacuum to yield 8.1 g. crude TBDMS-Vitamin-K2-5 as oil. The crude product was purified by column chromatography using mixture of hexane and ethyl acetate to yield 5.2 g. TBDMS-Vitamin-K2-5 as oil.

### Example 8

Synthesis of TBDMS-Vitamin-K2-6 (C₅₃H₈₆O₂Si₂) (811.44)

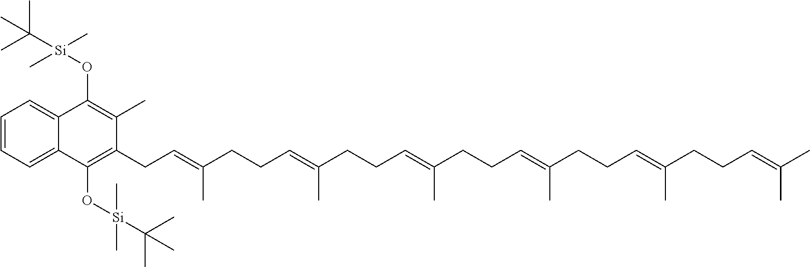 [SMILES: CC(C)=CCC/C(C)=C/CC/C(C)=C/CC/C(C)=C/CC/C(C)=C/CC/C(C)=C/Cc1c(C)c(O[Si](C)(C)C(C)(C)C)c2ccccc2c1O[Si](C)(C)C(C)(C)C]

6.11 g. (˜0.01 mol) TBDMS-Vitamin-K2-1-SO₂Ph was dissolved in 21 ml tetrahydrofuran and 1.85 ml dimethyl formamide under inert atmosphere. 4.71 g. (˜0.0125 mol) penta prenyl chloride was added. The reaction mass was cooled to 0 to −5° C. 0.266 g. (˜0.000825 mol) tetra butyl ammonium bromide and 0.044 g (˜0.000165 mol) 18 crown 6 were added. The reaction mass was stirred at the same temperature for 5 minutes. 1.68 g (0.015 mol) potassium tert-butoxide in 16 ml tetrahydrofuran was added to above solution over 30 minutes and stirred at the same temperature for 2 hrs. After completion of the reaction, it was quenched by the dropwise addition of 20% ammonium chloride solution and the pH was adjusted to 5-6 using 1 M HCl. Tetrahydrofuran was distilled under vacuum and the residue was extracted in ethyl acetate. Ethyl acetate layer was washed with water followed by brine and dried over anhydrous sodium sulphate. Ethyl acetate was distilled under vacuum to yield 9.7 g. crude TBDMS-Vitamin-K2-6-SO₂Ph as oil. The crude product was purified by column chromatography using mixture of hexane and ethyl acetate to yield 6.6 g. TBDMS-Vitamin-K2-6-SO₂Ph as oil. 9.51 g. (˜0.01 mol) TBDMS-Vitamin-K2-6-SO₂Ph was dissolved in 95 ml tetrahydrofuran under inert atmosphere and 0.01 g (˜0.000017 mol) (1,3-bis (diphenyl phosphino) propane) palladium II chloride was added. The reaction mass was cooled to −5° C. 3.18 g (0.03 mol) super hydride solution was added over 30 minutes. The reaction mass was stirred at room temperature for 10-12 hrs. After completion of the reaction, it was quenched by the dropwise addition of methanol followed by acetic acid and water. The reaction mass stirred for 3 hrs and extracted twice with ethyl acetate. Ethyl acetate layer was washed with water followed by brine and dried over anhydrous sodium sulphate. Ethyl acetate was distilled under vacuum to yield 8.9 g. crude TBDMS-Vitamin-K2-6 as oil. It was purified by column chromatography using mixture of hexane and ethyl acetate to yield 5.6 g. TBDMS-Vitamin-K2-6 as oil.

¹HNMR (CDCl₃): 8.01 (m), 2H, 7.36 (m), 2H, 5.13 (m), 3H, 3.52 (dd), 2H, 2.29 (m), 3H, 1.99-2.07 (m), 20H, 1.77 (s), 3H, 1.73 (s), 3H, 1.61 (m), 6H, 1.13 (m), 18H, 0.92 (s), 3H, 0.16 (dd), 12H, ¹³CMR (CDCl₃): 143.07, 142.47, 135.16, 135.03, 134.91, 134.90, 131.27, 127.60, 127.20, 127.04, 124.43, 124.31, 124.29, 124.25, 124.16, 124.09, 123.90, 123.45, 122.98, 122.68, 39.76, 39.62, 31.97, 29.75, 29.71, 29.41, 27.07, 26.78, 26.73, 26.69, 26.25, 26.18, 25.75, 25.73, 25.68, 22.74, 18.77, 18.72, 18.16, 17.72, 16.48, 16.04, 14.45, 14.18, −2.90, −2.98, −3.01, −3.14, −3.21.

Mass: 808 (m-3).

### Example 9

Synthesis of TBDMS-Vitamin-K2-7 (C₅₈H₉₄O₂Si₂) (879.55)

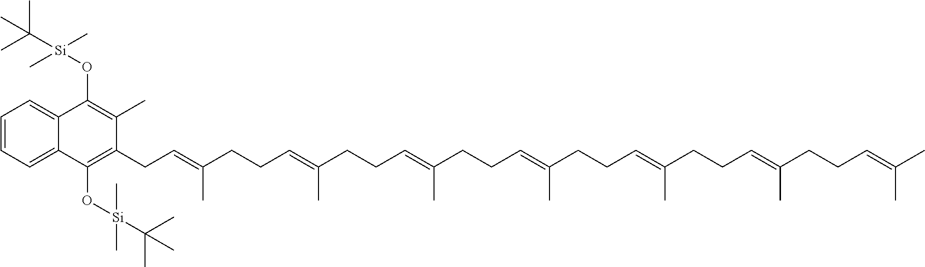 [SMILES: CC(C)=CCC/C(C)=C/CC/C(C)=C/CC/C(C)=C/CC/C(C)=C/CC/C(C)=C/CC/C(C)=C/Cc1c(C)c(O[Si](C)(C)C(C)(C)C)c2ccccc2c1O[Si](C)(C)C(C)(C)C]

6.11 g. (˜0.01 mol) TBDMS-Vitamin-K2-1-SO₂Ph was dissolved in 21 ml tetrahydrofuran and 1.85 ml dimethyl formamide under inert atmosphere. 5.56 g. (˜0.0125 mol) hexaprenyl chloride was added. The reaction mass was cooled to 0 to −5° C. 0.266 g. (˜0.000825 mol) tetra butyl ammonium bromide and 0.044 g. (˜0.000165 mol) 18 crown 6 were added. The reaction mass was stirred at the same temperature for 5 minutes. 1.68 g. (0.015 mol) potassium tert-butoxide in 16 ml tetrahydrofuran was added to the above solution over 30 minutes and stirred at the same temperature for 2 hrs. After completion of the reaction, it was quenched by the dropwise addition of 20% ammonium chloride solution and pH was adjusted to 5-6 using 1 M HCl. Tetrahydrofuran was distilled under vacuum and the residue was extracted in ethyl acetate. Ethyl acetate layer was washed with water followed by brine and dried over anhydrous sodium sulphate. Ethyl acetate was distilled under vacuum to yield 10.5 g. crude TBDMS-Vitamin-K2-7-SO₂Ph as oil. It was purified by column chromatography using mixture of hexane and ethyl acetate to yield 7.1 g. TBDMS-Vitamin-K2-7-SO₂Ph as oil.

10.19 g. (˜0.01 mol) TBDMS-Vitamin-K2-7-SO₂Ph was dissolved in 101 ml tetrahydrofuran under inert atmosphere and 0.01 g (˜0.000017 mol) (1, 3-bis (diphenyl phosphino) propane) palladium II chloride was added. The reaction mass was cooled to −5° C. 3.18 g. (0.03 mol) super hydride solution was added over 30 minutes. The reaction mass was stirred at room temperature for 10-12 hrs. After the completion of the reaction, it was quenched by dropwise addition of methanol followed by acetic acid and water. The reaction mass was stirred for 3 hrs and extracted twice with ethyl acetate. Ethyl acetate layer was washed with water followed by brine and dried over anhydrous sodium sulphate. Ethyl acetate was distilled under vacuum to yield 9.7 g. crude TBDMS-Vitamin-K2-7 as oil. The crude product was purified by column chromatography using mixture of hexane and ethyl acetate to yield 6.1 g. TBDMS-Vitamin-K2-7 as oil.

¹HNMR (CDCl₃): 8.01 (m), 2H, 7.36 (m), 2H, 5.0 (m), 1H, 5.12 (m), 6H, 3.51 (m), 2H, 2.28 (s), 3H, 1.98-2.06 (m), 12H, 1.60 (m), 24H, 1.0 (s), 18H, 0.08 (d), 12H. ¹³CMR (CDCl₃): 143.09, 142.48, 135.16, 135.03, 134.91, 131.24, 127.59, 127.20, 127.04, 124.43, 124.30, 124.25, 124.16, 124.07, 123.88, 123.46, 122.98, 122.63, 43.53, 39.76, 39.62, 36.30, 34.23, 31.96, 29.74, 29.69, 29.40, 27.08, 26.79, 26.77, 26.71, 26.24, 26.18, 25.73, 22.73, 18.77, 18.72, 17.71, 16.46, 16.03, 16.01, 14.44, 14.15, −3.15, −3.22. Mass: 880 (m+1).

### Example 10

Synthesis of TBDMS-Vitamin-K2-9: (C₆₈H₁₁₀O₂Si₂) (1015.79)

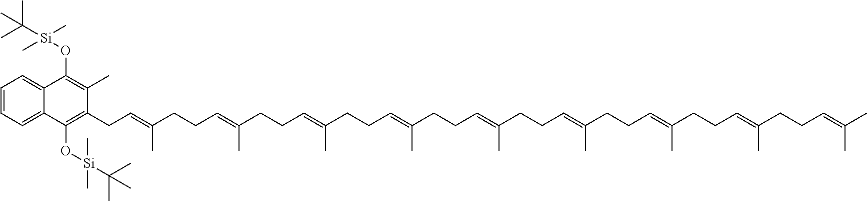 [SMILES: CC(C)=CCC/C(C)=C/CC/C(C)=C/CC/C(C)=C/CC/C(C)=C/CC/C(C)=C/CC/C(C)=C/CC/C(C)=C/CC/C(C)=C/Cc1c(C)c(O[Si](C)(C)C(C)(C)C)c2ccccc2c1O[Si](C)(C)C(C)(C)C]

6.11 g. (˜0.01 mol) TBDMS-Vitamin-K2-1-SO₂Ph was dissolved in 21 ml tetrahydrofuran and 1.85 ml dimethyl formamide under inert atmosphere. 7.26 g. (˜0.0125 mol) octa prenyl chloride was added. The reaction mass was cooled to 0 to −5° C. 0.266 g. (˜0.000825 mol) tetra butyl ammonium bromide and 0.044 g. (˜0.000165 mol) 18 crown 6 were added. The reaction mass was stirred at the same temperature for 5 minutes. 1.68 g. (0.015 mol) potassium tert-butoxide in 16 ml tetrahydrofuran was added to the above solution over 30 minutes and stirred at the same temperature for 2 hrs. After completion of the reaction, it was quenched by the dropwise addition of 20% ammonium chloride solution and the pH was adjusted to 5-6 using 1 M HCl. Tetrahydrofuran was distilled under vacuum and the residue was extracted in ethyl acetate. Ethyl acetate layer was washed with water followed by brine and dried over anhydrous sodium sulphate. Ethyl acetate was distilled under vacuum to yield 13 g. crude TBDMS-Vitamin-K2-9-SO₂Ph as oil. The crude product was purified by column chromatography using mixture of hexane and ethyl acetate to yield 8 g. TBDMS-Vitamin-K2-9-SO₂Ph as oil. 11.55 g. (˜0.01 mol) TBDMS-Vitamin-K2-9-SO₂Ph was dissolved in 115 ml tetrahydrofuran under inert atmosphere and 0.01 g. (˜0.000017 mol) (1,3-bis (diphenyl phosphino) propane) palladium II chloride was added. The reaction mass was cooled to −5° C. 3.18 g. (0.03 mol) super hydride solution was added in 30 minutes. The reaction mass was stirred at room temperature for 10-12 hrs. After completion of the reaction, it was quenched by dropwise addition of methanol followed by acetic acid and water. The reaction mass was stirred for 3 hrs and extracted twice with ethyl acetate. Ethyl acetate layer was washed with water followed by brine and dried over anhydrous sodium sulphate. Ethyl acetate was distilled under vacuum to yield 10.4 g. crude TBDMS-Vitamin-K2-9 as oil. It was purified by column chromatography using mixture of hexane and ethyl acetate to yield 7.1 g. TBDMS-Vitamin-K2-9 as oil.

### Example 11

### Oxidation of TBDMS Ethers of Vitamin K2-1-SO2Ph to Carbonyl Compounds

CrO₃ 20 mg (0.2 mmol) and H₅IO₆ 1.09 g. (4.781 mmol) were dissolved in acetonitrile (4 ml) by vigorous stirring at room temperature for 20 minutes. The solution was added slowly into the pre cooled solution of 1 g. (1.636 mmol) TBDMS-Vitamin-K2-1-SO2Ph in 5 ml methylene chloride at −5 to 0° C. in 10-15 minutes. After the addition was complete, the reaction mixture was stirred at −5 to 0° C. for an additional 1 hr, quenched with saturated aqueous Na₂S₂O₃ solution (10 mL) and then filtered. The filtrate was concentrated under vacuum and the residue was diluted with ethyl acetate (20 mL), washed with water (10 ml), saturated aqueous Na₂S₂O₃ solution (10 ml), brine (10 ml), and then dried over Na₂SO₄ (1 g.). The solvent was removed under reduced pressure and the residue was purified by flash chromatography (hexane-ethyl acetate 2:8) to yield K2-1-SO2Ph.

¹HNMR (CDCl₃): 7.96-8.03 (m) 2H; 7.67 (m) 4H; 7.23 (m) 3H; 4.82 (m) 1H; 3.72 (s) 2H; 3.22 (D) 2H; 1.98 (s) 3H; 1.92 (s) 3H. ¹³CMR (CDCl₃): 184.93, 183.82, 144.05, 143.58, 137.79, 133.56, 133.32, 131.91, 131.81, 130.63, 128.75, 128.29, 126.22, 126.20, 125.86, 65.80, 26.27, 16.96, 12.71. Mass: 381.4 (m+1).

### Example 12

### Oxidation of Compound (TBDMS-K2-6) to Vitamin K2-6

CrO₃ 10 mg (0.1 mmol) and H₅IO₆ 0.421 g. (1.84 mmol) were dissolved in acetonitrile (5 ml) by vigorous stirring at room temperature for 20 minutes. The solution was added slowly into the pre cooled solution of 0.5 g. (0.616 mmol) TBDMS-Vitamin-K2-6 in 5 ml methylene chloride at −5 to 0° C. in 10-15 minutes. After the addition was complete, the reaction mixture was stirred at −5 to 0° C. for an additional 1 hr, quenched with saturated aqueous Na₂S₂O₃ solution (10 ml) and filtered. The filtrate was washed with water (10 ml), brine (10 ml), and then dried over Na₂SO₄ (1 g.). The solvent was removed under reduced pressure and the residue was purified by flash chromatography (hexane-ethyl acetate 2:8 v/v) to yield Vitamin K2-6.

¹HNMR (CDCl₃): 8.10 (m), 2H, 7.71 (m), 2H, 5.12 (m), 6H, 3.39 (D), 2H, 2.20 (s), 3H, 1.93-2.07 (m), 20H, 1.81 (s), 3H, 1.69 (s), 3H, 1.52-1.61 (d), 15H. ¹³CMR (CDCl₃): 185.26, 184.34, 146.06, 143.27, 137.45, 135.14, 134.84, 134.80, 133.25, 133.19, 132.13, 132.10, 131.13, 126.26, 126.14, 124.43, 124.28, 124.16, 123.86, 119.12, 39.73, 39.68, 26.76, 26.68, 26.65, 26.63, 26.48, 25.99, 25.70, 17.67, 16.40, 16.03, 16.00, 15.98, 12.64. Mass: 581.19 (m+1).

### Oxidation of Compound (TBDMS-K2-7) to Vitamin K2-7

CrO₃ 20 mg (0.2 mmol) and H₅IO₆ 0.777 g. (3.41 mmol) were dissolved in acetonitrile (5 ml) by vigorous stirring at room temperature for 20 minutes. The solution was added slowly into the pre cooled solution of 1 g. (1.13 mmol) TBDMS-Vitamin-K2-7 in 5 ml methylene chloride at 0 to −5° C. in 10-15 minutes. After the addition was complete, the reaction mixture was stirred at 0 to −5° C. for an additional 1 hr, quenched with saturated aqueous Na₂S₂O₃ solution (10 ml) and then filtered. The filtrate was washed with water (10 ml), brine (10 ml), and then dried over Na₂SO₄ (1 g.). The solvent was removed under reduced pressure and the residue was purified by flash chromatography (hexane-ethyl acetate 2:8 v/v) to yield Vitamin K2-7.

¹HNMR (CDCl₃): 8.11 (m), 2H, 7.71 (m), 2H, 5.12 (m), 7H, 3.40 (D), 2H, 2.21 (s), 3H, 1.93-2.07 (m), 24H, 1.81 (s), 3H, 1.70 (s), 3H, 1.52-1.61 (d), 18H. ¹³CMR (CDCl₃): 185.34, 184.41, 146.10, 143.30, 137.49, 135.17, 134.86, 133.28, 133.23, 132.15, 132.12, 131.18, 126.28, 126.17, 124.42, 124.27, 124.15, 123.85, 119.10, 39.73, 39.69, 26.77, 26.69, 26.66, 26.48, 26.00, 25.71, 17.68, 16.42, 16.02, 12.66. Mass: 649.81 (m+1).

### Comparative Examples

6.07 g. (0.01 mol) TBDMS K2-3 was dissolved in 25 ml tetrahydrofuran and 7.83 g (0.03 mole) tetra butyl ammonium fluoride (TBAF) was added at room temperature. The reaction was monitored by thin layer chromatography. TBDMS K2-3 was completely reacted in 2 hrs as indicated by thin layer chromatography but the desired product was not formed, instead TBDMS K2-3 was hydrolysed.

6.07 g. (0.01 mol) TBDMS K2-3 was dissolved in 25 ml 50% aqueous methanol and 9.22 g. (0.03 mol) Oxone (potassium peroxy mono sulphate) was added in portions over 30 minutes at room temperature. The reaction was monitored by thin layer chromatography. Even at the end of 24 hrs only TBDMS K2-3 was found to be present indicating the oxidation did not proceed.

6.07 g. (0.01 mol) TBDMS K2-3 was dissolved in 25 ml methanol and 2.4 g. (0.005 mol) tetra butyl ammonium tribromide (TBATB) was added at room temperature. The reaction was continued for 24 hrs at room temperature and monitored by thin layer chromatography. At the end of 24 hrs only the starting compound TBDMS K2-3 was present. The temperature of the reaction mass was raised to 50° C. and the reaction was further monitored for 4-5 hrs by thin layer chromatography. At the end of 5 hrs only the starting compound TBDMS K2-3 was present indicating that the oxidation reaction did not take place.

6.07 g. (0.01 mol) TBDMS K2-3 was dissolved in 25 ml acetonitrile and 2.4 g (0.005 mol) tetra butyl ammonium tribromide (TBATB) at room temperature was added. The reaction was continued for 24 hrs at room temperature and monitored by thin layer chromatography. Even after 24 hrs only starting compound TBDMS K2-3 was found to be present indicating thereby that the oxidation reaction had not taken place.

6.07 g. (0.01 mol) TBDMS K2-3 was dissolved in 25 ml tetrahydrofuran and 2.4 g. (0.005 mol) tetra butyl ammonium tribromide (TBATB) at room temperature was added. The reaction was continued for 24 hrs at room temperature and monitored by thin layer chromatography. Even after 24 hrs only the starting compound TBDMS K2-2 was found to be present. 25 ml methanol was added and the reaction was continued at 50° C. and monitored for 4-5 hrs by thin layer chromatography. Even after 5 hrs only the starting compound TBDMS K2-3 was found to be present indicating that the oxidation reaction did not take place.

6.07 g. (0.01 mol) TBDMS K2-3 was dissolved in 25 ml methanol and cooled to 0-5° C. 2.35 g. (0.03 mol) acetyl chloride was added dropwise over 10-15 mins. The reaction was continued at 0-5° C. for 1-2 hrs and monitored by thin layer chromatography. Even after 24 hrs only starting compound was present indicating that oxidation reaction did not take place.

6.07 g. (0.01 mol) TBDMS K2-3 was dissolved in 25 ml methanol and 0.55 g. (0.0015 mol) tetra butyl ammonium iodide (TBAI) at room temperature was added. The reaction was monitored by thin layer chromatography. Even after 24 hrs only starting compound TBDMS K2-3 was present indicating that the oxidation reaction did not take place.

Oxidation of Compound (Dimethyl K2-7) to Vitamin K2-7 CrO₃ 20 mg (0.2 mmol) and H₅IO₆ 1 g. (4.4 mmol) were dissolved in acetonitrile (20 ml) by vigorous stirring at room temperature for 20 minutes. The solution was added slowly into the pre cooled solution of 1 g. (1.47 mmol) dimethyl K2-7 in 20 ml methylene chloride at −5 to 0° C. in 10-15 minutes. After the addition was complete, the reaction mixture was stirred at −5 to 0° C. for an additional 1 hr. The reaction was monitored by TLC. The reaction did not proceed.

### Oxidation of Compound (Dibenzyl K2-3) to Vitamin K2-3

CrO₃ 20 mg (0.2 mmol) and H₅IO₆ 1.22 g. (5.37 mmol) were dissolved in acetonitrile (20 ml) by vigorous stirring at room temperature for 20 minutes. The solution was added slowly into the pre cooled solution of 1 g. (1.79 mmol) dibenzyl K2-3 in 20 ml methylene chloride at −5 to 0° C. in 10-15 minutes. After the addition was complete, the reaction mixture was stirred at −5 to 0° C. for an additional 1 hr. The reaction was monitored by TLC. The reaction did not proceed.

### Oxidation of Compound (Dimethyl K2-3) to Vitamin K2-3

CrO₃ 30 mg (0.3 mmol) and H₅IO₆ 2.48 g. (10.8 mmol) were dissolved in acetonitrile (20 ml) by vigorous stirring at room temperature for 20 minutes. The solution was added slowly into the pre cooled solution of 1 g. (2.4 mmol) dimethyl K2-3 in 20 ml methylene chloride at −5 to 0° C. in 10-15 minutes. After the addition was complete, the reaction mixture was stirred at −5 to 0° C. for an additional 1 hr. The reaction was monitored by TLC. The reaction did not proceed.

### Example 13

2 g (0.0033 mol) TBDMS-K2-1-SO2Ph was dissolved in 20 ml methylene chloride. The reaction mass was cooled to 0 to −5° C. 2.2 g. (0.0098 mol) periodic acid and 40 mg chromium trioxide were dissolved in 20 ml acetonitrile and added to the reaction mass over 5 minutes. The reaction mass was stirred at 0 to −5° C. and the reaction was monitored at specific time intervals and finally after carrying out overnight at room temperature, using thin layer chromatography and high-pressure liquid chromatography. The reaction was quenched by 10% sodium thiosulphate solution and the reaction mass was filtered through the celite bed. Aqueous layer was further extracted with methylene chloride. The organic layer was washed twice with water followed by brine solution and dried over anhydrous sodium sulphate. The solvent was stripped off to recover the product. Reaction was carried out varying CrO3 wt. % based on TBDMS-K2-1-SO2Ph and varying mole ratio of TBDMS-K2-1-SO2Ph to Periodic acid. Values of % Conversion vs Time at various time intervals is summarized below.

| | TBDMS-K2-1- | | | | | | |
|---|---|---|---|---|---|---|---|
| Sr | SO2Ph:Periodic | CrO3 | | | | | Over |
| No | acid | wt. % | 30 min | 1 hr | 2 hr | 4 hr | night |
| 1 | 1:3 | 2 | 95.21 | 94.63 | 95.36 | 93.40 | — |
| 2 | 1:3 | 1.5 | 94.28 | 94.33 | 94.17 | 93.29 | 76.75 |
| 3 | 1:4 | 0.5 | 90.93 | 89.30 | 88.44 | 92.85 | 41.75 |
| 4 | 1:2 | 2 | 87.94 | 82.43 | 93.99 | 92.94 | 82.55 |

1-16. (canceled)

17: A process for the condensation of phenyl sulphone of protected mono prenyl menadiol of the formula (I) with a prenyl halide of formula (II) 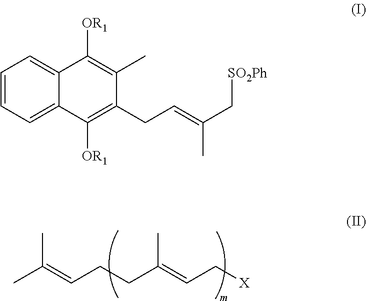
wherein R₁ is a protecting group selected from the group consisting of trimethylsilyl (TMS), t-butyldimethylsilyl (TBDMS), and tri isopropyl silyl (TIPS) ethers;
m is selected from 0 to 7;
X is halogen atom selected from chlorine and bromine; 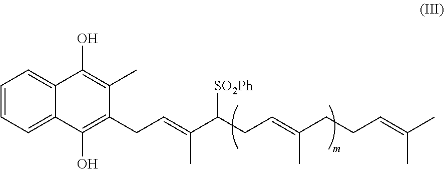
to yield the phenyl sulphonyl derivative of menadiol of the formula (III) in the presence of a strong organometallic base and phase transfer catalysts comprising a quaternary alkyl ammonium halide and a crown ether.

18: The process of claim 17, wherein the mole ratio of phenylsulfone of protected mono prenyl menadiol to prenyl halide is in the range 1:1.25 to 1:1.33.

19: The process of claim 17, wherein the reaction is carried out in the temperature range −5 to 0° C.

20: The process of claim 17, wherein the quaternary alkyl ammonium halide is tetra butyl ammonium bromide (TBAB).

21: The process of claim 17, wherein the crown ether is 1, 4, 7, 10, 13, 16-hexa oxacyclo octadecane.

22: The process of claim 17, wherein the mole ratio of tetra butyl ammonium bromide to 1, 4, 7, 10, 13, 16-hexa oxacyclo octadecane is in the range 0.5 to 20.

23: The process of claim 17, wherein the reaction time is in the range 0.25 to 6 hours.

24: A process for the oxidation of the silyl protected Vitamin K2 in the presence of chromium trioxide and periodic acid to yield Vitamin K2, wherein the Vitamin K2 is selected from Vitamin K2-2 to K2-9 and the silyl protecting group is selected from trimethylsilyl (TMS), t-butyldimethylsilyl (TBDMS), and tri isopropyl silyl (TIPS) group.

25: The process of claim 24, wherein the mole ratio of periodic acid to the silyl protected Vitamin K2 is in the range 2:1 to 4:1.

26: The process of claim 24, wherein the ratio of chromium trioxide to the silyl protected Vitamin K2 is in the range 0.5 to 2 wt. %.

27: The process of claim 24, wherein the oxidation is carried out in the temperature range −5 to 0° C.

28: A process for the synthesis of Vitamin K2 represented by the formula (IV): 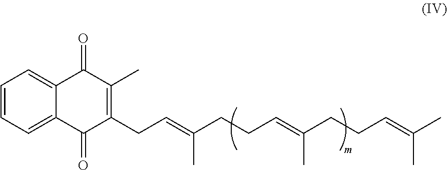
wherein m is selected from 0 to 7 comprising the steps of
(a) reacting the phenylsulfone of monoprenyl menadiol derivative of formula (I): 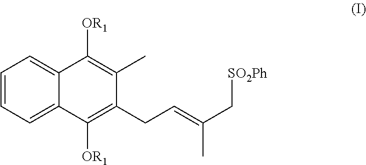
wherein R₁ represents a protecting group selected from the group consisting of trimethylsilyl (TMS), t-butyldimethylsilyl (TBDMS), and tri isopropyl silyl (TIPS) ether, in the presence of a strong organometallic base and phase transfer catalysts comprising a quaternary alkyl ammonium halide and a crown ether with a prenyl halide of the formula (II): 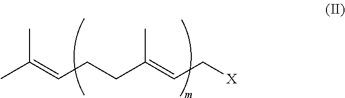
to yield the phenyl sulphonyl derivative of menadiol of the formula (III): 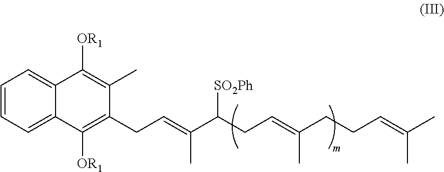
b) removing the phenyl sulphonyl group from the compound of the formula (III) by the reductive elimination in the presence of [1,2-bis (diphenyl phosphino) ethane] dichloro palladium (II) and lithium triethyl borohydride to yield the menadiol derivative of the formula (V): 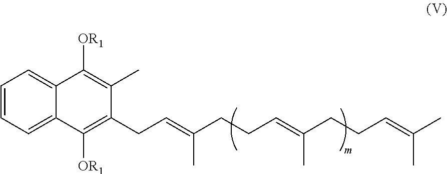
wherein m is selected from 0 to 7;
c) subjecting the menadiol derivative of the formula (V) to an oxidative deetherification in the presence of chromium trioxide and periodic acid to yield Vitamin K2 of the formula (IV): 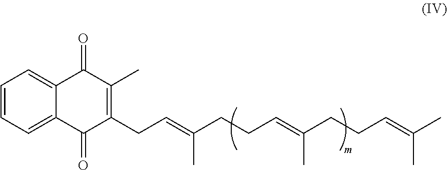
and optionally purifying the crude product to obtain pure Vitamin K2.

29: The process of claim 28, wherein the phenyl sulphone of protected mono prenyl menadiol of the formula (I) is obtained from the bromo derivative of protected monoprenyl menadiol by reacting the said derivative in the presence of activated magnesium with chloro prenyl sulphonate, catalysed by cuprous bromide in a polar solvent.

30: A compound of the formula (I): 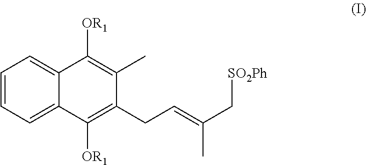
wherein R₁ represents a protecting group selected from the group consisting of trimethylsilyl (TMS) t-butyldimethylsilyl (TBDMS), and tri isopropyl silyl (TIPS) ether.

31: A compound of the formula (III): 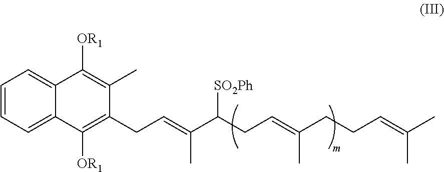
wherein R₁ is a protecting group, selected from the group consisting of trimethylsilyl (TMS), t-butyldimethylsilyl (TBDMS), and tri isopropyl silyl (TIPS) ethers and m is in the range 0 to 7.

32: A compound of the formula (V): 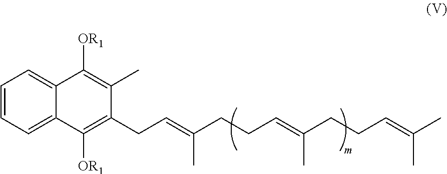
wherein R₁ is protecting group selected from the group consisting of trimethylsilyl (TMS), t-butyldimethylsilyl (TBDMS), and tri isopropyl silyl (TIPS) ethers and m is in the range 0 to 7.
